(19) United States
(12) Patent Application Publication
(10) Pub. No.: US 2020/0368396 A1
(43) Pub. Date: Nov. 26, 2020

(54) PREVENTION OF CARTILAGE DEGENERATION SURROUNDING FOCAL CHONDRAL DEFECTS

(71) Applicant: The Regents of the University of Colorado, a body corporate, Denver, CO (US)

(72) Inventors: Stephanie J. Bryant, Boulder, CO (US); Sarah Schoonraad, Longmont, CO (US); Elizabeth A. Aisenbrey, Madison, WI (US)

(21) Appl. No.: 16/868,863

(22) Filed: May 7, 2020

Related U.S. Application Data
(60) Provisional application No. 62/844,511, filed on May 7, 2019.

(51) Int. Cl. A61L 27/54; A61L 27/52; C08L 67/02

(52) U.S. Cl. CPC A61L 27/54; A61L 2300/252; C08L 67/02; A61L 27/52

(57) ABSTRACT

The field of the invention is directed to methods for treating cartilage disorders, diseases and injuries including, but not limited to, focal chondral defects. The field of the invention is also directed to methods for preventing cartilage disorders and diseases including, but not limited to, degenerative disc disease or osteoarthritis. The field of the invention is further directed to reducing inflammation associated with cartilage disorders, diseases and injuries including, but not limited to, focal chondral defects, degenerative disc disease, or osteoarthritis. The field of the invention is also directed to compositions useful in the methods for treatment of focal chondral defects and/or reducing inflammation associated with disorders, diseases and injuries including, but not limited to focal chondral defects, degenerative disc disease, or osteoarthritis.

### CROSS-REFERENCE TO RELATED APPLICATIONS

The present application claims the benefit of U.S. Provisional Patent Application No. 62/844,511, filed on May 7, 2019, which is incorporated herein by reference.

### STATEMENT OF GOVERNMENTAL SUPPORT

This invention was made with government support under grant number AR069060 awarded by the National Institutes of Health. The government has certain rights in the invention.

### FIELD OF THE INVENTION

The field of the invention is directed to methods for treating cartilage disorders, diseases and injuries including, but not limited to, focal chondral defects. The field of the invention is also directed to methods for preventing cartilage disorders and diseases including, but not limited to, degenerative disc disease or osteoarthritis. The field of the invention is further directed to reducing inflammation associated with cartilage disorders, diseases and injuries including, but not limited to, focal chondral defects, degenerative disc disease, or osteoarthritis. The field of the invention is also directed to compositions useful in the methods for treatment of focal chondral defects and/or reducing inflammation associated with disorders, diseases and injuries including, but not limited to focal chondral defects, degenerative disc disease, or osteoarthritis.

### BACKGROUND OF THE INVENTION

Cartilage is a flexible connective tissue found in the joints between bones, the rib cage, the ear, the nose, the bronchial tubes, the pubic symphysis, and the intervertebral discs. Cartilage is not as hard and rigid as bone but is stiffer and less flexible than tendons and ligaments. Cartilage is made by specialized cells called chondroblasts that produce a large amount of extracellular matrix composed of collagen fibers, abundant ground substance rich in proteoglycan, and elastin fibers. Cartilage is classified in three types, elastic cartilage, hyaline cartilage and fibrocartilage, which differ in the relative amounts of these three main components. Chondroblasts that get caught in the matrix are called chondrocytes. They reside in spaces called lacunae with up to eight chondrocytes per lacuna.

Unlike other connective tissues, cartilage does not contain blood vessels. The chondrocytes are supplied by diffusion, which is helped by the pumping action generated by compression of the articular cartilage or flexion of the elastic cartilage. Because it does not have a direct blood supply, compared to other connective tissues, cartilage grows and repairs much more slowly. As a result, when cartilage is injured or diseased, it is very difficult to heal. It is believed that a treatment option that could prevent the development of cartilage disorders and diseases, accelerate healing of cartilage once injured or diseased, perhaps eliminating the need for surgical intervention in severe cases, is desirable. Accordingly, it is an object of the instant invention to provide such a treatment option to subjects suffering from cartilage diseases, disorders and injuries including, but not limited to, focal chondral defects, degenerative disc disease, or osteoarthritis.

### SUMMARY OF THE INVENTION

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The field of the invention is directed to methods for treating cartilage disorders, diseases and injuries including, but not limited to, focal chondral defects. The field of the invention is also directed to methods for preventing cartilage disorders and diseases including, but not limited to, degenerative disc disease or osteoarthritis. The field of the invention is further directed to reducing inflammation associated with cartilage disorders, diseases and injuries including, but not limited to, focal chondral defects, degenerative disc disease, or osteoarthritis. The field of the invention is also directed to compositions useful in the methods for treatment of focal chondral defects and/or reducing inflammation associated with disorders, diseases and injuries including, but not limited to focal chondral defects, degenerative disc disease, or osteoarthritis.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Other objects, advantages, and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

In one embodiment, the invention relates to a method for treating a chondral defect in a patient in need thereof comprising administering to the patient a therapeutically effective amount of a hydrogel composition within said chondral defect. In one embodiment, said treating prevents cartilage diseases. In one embodiment, said cartilage disease comprises joint degeneration. In one embodiment, said cartilage disease comprises osteoarthritis. In one embodiment, said chondral defect is due to an injury. In one embodiment, said treating reduces the effects of cartilage degneration. In one embodiment, said treating comprises infilling said chondral defect with said hydrogel composition. In one embodiment, said hydrogel composition produce swelling pressures of 13 and 310 kPa. In one embodiment, said hydrogel composition produce swelling pressures of 13 and 310 kPa. In one embodiment, said treating reduces the loss of sulfated glycosaminoglycans surrounding said chondral defect. In one embodiment, said hydrogel is polymerized in situ. In one embodiment, said hydrogel comprises Poly(ethylene glycol) (PEG). In one embodiment, said hydrogel comprises Poly(ethylene glycol) dimethacrylate. In one embodiment, said treating comprises integration of a 3D-scaffold into said chondral defect in addition to said hydrogel composition. In one embodiment, hydrogel composition demonstrates tunability of hydrogel to control volume expansion. In one embodiment, said hydrogel composition demonstrates the ability to combine swelling pressure of soft gel with 3D-printed stiff structures. In one embodiment, said hydrogel composition comprises a polymer weight percentage 5% (20 kDa) (MW PEG) with a ratio of thiol:ene of 0.95. In one embodiment, said hydrogel composition comprises a polymer weight percentage 10% (10 kDa) (MW PEG) with a ratio of thiol:ene of 1.00. In one embodiment, said hydrogel composition comprises a polymer weight percentage 10% (10 kDa) (MW PEG) with a ratio of thiol:ene of 0.90. In one embodiment, said hydrogel composition comprises a polymer weight percentage 10% (10 kDa) (MW PEG) with a ratio of thiol:ene of 0.65. In one embodiment, said hydrogel composition comprises a polymer weight percentage 8% (20 kDa) (MW PEG) with a ratio of thiol:ene of 0.95. In one embodiment, said hydrogel composition comprises a polymer weight percentage 7% (10 kDa) (MW PEG) with a ratio of thiol:ene of 0.90. In one embodiment, said hydrogel composition comprises a polymer weight percentage 15% (10 kDa) (MW PEG) with a ratio of thiol:ene of 0.75. In one embodiment, said hydrogel composition comprises a polymer weight percentage 9% (20 kDa) (MW PEG) with a ratio of thiol:ene of 0.90. In one embodiment, said hydrogel composition comprises a polymer weight percentage 9% (20 kDa) (MW PEG) with a ratio of thiol:ene of 1.00. In one embodiment, said hydrogel composition comprises a polymer weight percentage 10% (10 kDa) (MW PEG) with a ratio of thiol:ene of 0.50. In one embodiment, said hydrogel composition comprises a polymer weight percentage 10% (20 kDa) (MW PEG) with a ratio of thiol:ene of 0.95. In one embodiment, said hydrogel composition comprises a polymer weight percentage 15% (10 kDa) (MW PEG) with a ratio of thiol:ene of 0.90. In one embodiment, said hydrogel composition comprises a polymer weight percentage 20% (10 kDa) (MW PEG) with a ratio of thiol:ene of 0.90. In one embodiment, said hydrogel composition comprises a polymer weight percentage 9% (20 kDa) (MW PEG) with a ratio of thiol:ene of 0.70. In one embodiment, said hydrogel composition comprises a polymer weight percentage 12% (20 kDa) (MW PEG) with a ratio of thiol:ene of 0.95. In one embodiment, said hydrogel composition comprises a polymer weight percentage 25% (10 kDa) (MW PEG) with a ratio of thiol:ene of 1.00. In one embodiment, said hydrogel composition comprises a polymer weight percentage 9% (20 kDa) (MW PEG) with a ratio of thiol:ene of 0.50. In one embodiment, said hydrogel composition comprises a polymer weight percentage 13% (20 kDa) (MW PEG) with a ratio of thiol:ene of 0.95. In one embodiment, said hydrogel composition comprises a polymer weight percentage 9% (20 kDa) (MW PEG) with a ratio of thiol:ene of 0.30. In one embodiment, said hydrogel comprises a polymer weight percentage between 5-10% 10-20 kDa MW PEG with a ratio of thiol:ene between 0.30-.95. In one embodiment, said hydrogel comprises a polymer weight percentage between 10-25% 10-20 kDa MW PEG with a ratio of thiol:ene between 0.50-1.00. In one embodiment, hydrogel PEG polymers are created by photopolymerization of mixtures of multifunctional thiols and enes, such as described by Hoyle [25] and Lin [26].

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As defined herein “isolated” refers to material removed from its original environment and is thus altered “by the hand of man” from its natural state.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

By the term “animal-free” when referring to certain compositions, growth conditions, culture media, etc. described herein, is meant that no non-human animal-derived materials, such as non-human animal-derived serum, other than clinical grade human materials, such as recombinantly produced human proteins, are used in the preparation, growth, culturing, expansion, storage or formulation of the certain composition or process.

By the term “serum-free” when referring to certain compositions, growth conditions, culture media, etc. described herein, is meant that no animal-derived serum (i.e. no non-human animal) is used in the preparation, growth, culturing, expansion, storage or formulation of the certain composition or process.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of cells in the primary culture after 5 passages, as compared to about a 20-fold increase in such cells using previous methods. In another example, the level of cells per gram of amniotic tissue in expanded compositions of AMP cells is at least 30- and up to 100-fold higher than the number of cells in the primary culture after 3 passages. Accordingly, an “expanded” population has at least a 2-fold, and up to a 10-fold, improvement in cell numbers per gram of amniotic tissue over previous methods. The term “expanded” is meant to cover only those situations in which a person has intervened to elevate the number of the cells.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. As used herein, conditioned medium also refers to components, such as proteins, that are recovered and/or purified from conditioned medium or from ECS cells, including AMP cells.

As used herein, the term “cellular factor-containing solution” or “CFS” composition means a composition having physiologic concentrations of one or more protein factors. CFS compositions include conditioned media derived from ECS cells, amnion-derived cellular cytokine solution compositions (see definition below), physiologic cytokine solution compositions (see definition below), and sustained release formulations of such CFS compositions.

As used herein, the term “physiologic cytokine solution” or “PCS” composition means a composition which is not cell-derived and which has physiologic concentrations of VEGF, Angiogenin, PDGF and TGFβ2, TIMP-1 and TIMP-2.

As used herein, the term “suspension” means a liquid containing dispersed components, i.e. cytokines The dispersed components may be fully solubilized, partially solubilized, suspended or otherwise dispersed in the liquid. Suitable liquids include, but are not limited to, water, osmotic solutions such as salt and/or sugar solutions, cell culture media, and other aqueous or non-aqueous solutions.

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases.

The term “physiologic” or “physiological level” as used herein means the level that a substance in a living system is found and that is relevant to the proper functioning of a biochemical and/or biological process.

As used herein, the term “substrate” means a defined coating on a surface that cells attach to, grown on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

The term “therapeutically effective amount” means that amount of a therapeutic agent necessary to achieve a desired physiological effect (i.e. prevent or treat cartilage diseases, disorders and injuries).

As used herein, the term “pharmaceutically acceptable” means that the components, in addition to the therapeutic agent, comprising the formulation, are suitable for administration to the patient being treated in accordance with the present invention.

As used herein, the term “therapeutic component” means a component of the composition that exerts a therapeutic benefit when the composition is administered to a subject.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

As used herein, the term “tissue” refers to an aggregation of similarly specialized cells united in the performance of a particular function.

As used herein, the term “adjunctive” means jointly, together with, in addition to, in conjunction with, and the like.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, etc. By the term “inactive agent” is meant an agent that does not have a physiological effect when administered. Such agents may alternatively be called “pharmaceutically acceptable excipients”. Non-limiting examples include time release capsules and the like.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradeimal, intraperitoneal, transtracheal, subcutaneous, subcuticular, intra-articulare, subcapsular, subarachnoid, intraspinal, epidural, intracerebral, intraosseous, intracartilagenous, and intrasternal injection or infusion.

As used herein, the term “enteral” administration means any route of drug administration that involves absorption of the drug through the gastrointestinal tract. Enteral administration may be divided into three different categories, oral, gastric, and rectal. Gastric introduction involves the use of a tube through the nasal passage or a tube in the abdomen leading directly to the stomach.

As used herein, the term “topical” administration means a medication that is applied to body surfaces such as the skin or mucous membranes to treat ailments via a large range of classes including but not limited to liquids, creams, foams, gels, lotions, salves and ointments.

The terms “sustained-release”, “extended-release”, “time-release”, “controlled-release”, or “continuous-release” as used herein means an agent, typically a therapeutic agent or drug, that is formulated to dissolve slowly and be released over time.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, a “wound” is any disruption, from whatever cause, of normal anatomy (internal and/or external anatomy) including but not limited to traumatic injuries such as mechanical (i.e. contusion, penetrating, crush), thermal, chemical, electrical, radiation, concussive and incisional injuries; elective injuries such as operative surgery and resultant incisional hernias, fistulas, etc.; acute wounds, chronic wounds, infected wounds, and sterile wounds, as well as wounds associated with disease states (i.e. ulcers caused by diabetic neuropathy or ulcers of the gastrointestinal or genitourinary tract). A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scaring and/or the wounded area possesses tissue strength that is closer to that of uninjured tissue and/or the wounded tissue attains some degree of functional recovery.

As used herein the term “standard animal model” refers to any art-accepted animal model for in which the compositions of the invention exhibit efficacy.

As used herein the term “chondral defect” refers to a focal area of damage to the articular cartilage (the cartilage that lines the end of the bones). An osteochondral defect refers to a focal area of damage that involves both the cartilage and a piece of underlying bone.

As used herein the term “glycosaminoglycans” refers to long unbranched polysaccharides consisting of a repeating disaccharide unit. The repeating unit (except for keratan) consists of an amino sugar (N-acetylglucosamine or N-acetylgalactosamine) along with a uronic sugar (glucuronic acid or iduronic acid) or galactose. Glycosaminoglycans are highly polar and attract water. In a non-limiting example, they are therefore useful to the body as a lubricant or as a shock absorber.

### BRIEF DESCRIPTION OF THE DRAWINGS

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1A-B shows the evaluation of porcine cartilage adjacent to an empty focal chondral defect in vivo (FIG. 1A) and ex vivo (FIG. 1B). FIG. 1A) Representative images of Safranin-O/Fast green stained sections for sulfated glycosaminoglycans (sGAGs) surrounding the defect in vivo four weeks post-injury (i). A higher magnification of the boxed region is shown with arrows indicating decreased sGAGs (ii). FIG. 1B). Photographs of the ex vivo defect in osteochondral explants (i). Explants were cultured for four weeks under free swelling (FS) or under dynamic compressive loading (DL) (ii). Representative microscopy images for sGAGs are shown for full depth cartilage adjacent to the defect and width of sGAG loss was quantified (iii). The indentation modulus E* is presented (iv).

FIG. 2A shows a schematic and photographs of infilled focal chondral defects.

FIG. 2B shows a representative stress-strain plots (i) and compressive moduli (ii) for the soft and stiff hydrogels.

FIG. 2C shows temporal swelling behavior of the hydrogels is shown as a function of time under unconstrained conditions (i). Hydrogel volume increase from polymerized state to the equilibrium state is shown (ii).

FIG. 2D shows swelling pressures that were measured under constrained conditions as a function of time in soft and stiff hydrogels from the polymerized state to equilibrium (i). Maximum swelling pressures recorded at equilibrium are shown (ii).

FIG. 3A-D show an evaluation of cartilage adjacent to in situ filled focal chondral defects in the ex vivo porcine model under free swelling, FS (FIG. 3A and FIG. 3B) and under dynamic compressive loading, DL (FIG. 3C and FIG. 3D). Representative microscopy images are shown for full depth cartilage adjacent to the filled defect and width of sGAG loss was quantified (FIG. 3A and FIG. 3C). The indentation modulus E* is presented (FIG. 3B and FIG. 3D).

FIG. 4 shows the performance of the formulations 1-19 for swelling (as measured in volume increase %). The various formulations 1-19 are described in Table 1.

FIG. 5 shows an example of the integration of 3D-printed construct to provide support for physiological loads at injury site in combination with the tunable synthetic hydrogel.

FIG. 6A shows a porcine plugs with focal defect which was not treated with a hydrogel support. FIG. 6B shows a porcine plugs with focal defect which was treated with a hydrogel only support. FIG. 6C shows a porcine plugs with focal defect which was treated with a 3D-scaffold and hydrogel support.

### DETAILED DESCRIPTON OF THE INVENTION

Focal defects in articular cartilage are unable to self-repair and, if left untreated, are a leading risk factor for osteoarthritis. This study examined cartilage degeneration surrounding a defect and then assessed whether infilling the defect prevents degeneration. We created a focal chondral defect in porcine osteochondral explants and cultured them ex vivo with and without dynamic compressive loading to decouple the role of loading. When compared to a defect in a porcine knee four weeks post-injury, this model captured loss in sulfated glycosaminoglycans (sGAGs) along the defect's edge that was observed in vivo, but this loss was not load dependent. Loading, however, reduced the indentation modulus of the surrounding cartilage. After infilling with in situ polymerized hydrogels that were soft (100 kPa) or stiff (1 MPa) and which produced swelling pressures of 13 and 310 kPa, respectively, sGAG loss was reduced. This reduction correlated with increased hydrogel stiffness and swelling pressure, but was not affected by loading. This ex vivo model recapitulates sGAG loss surrounding a defect and, when infilled with a mechanically supportive hydrogel, degeneration is minimized.

### 1. Introduction

Acute injuries sustained to articulating joints can lead to focal defects in articular cartilage. Cartilage has a limited capacity to regenerate and on its own is unable to facilitate repair. When defects span into the underlying subchondral bone, the formation of a blood clot induces fibrocartilage repair. However, defects that are limited to the articular cartilage and left untreated remain empty. Since injury to articular cartilage is a leading risk factor for developing osteoarthritis [1], it is reasonable to postulate that a chondral defect in an otherwise healthy joint may represent a point source for early stages of cartilage degeneration. Several critical questions remain regarding the degeneration of cartilage tissue immediately surrounding an empty defect, such as the time course of degradation and whether degeneration can be prevented.

Chondral defects in articular cartilage are particularly vulnerable due to daily physical activity. Under mechanical loading, the tissue surrounding an empty chondral defect will be subjected to abnormal loads [2]. For example, an experimental whole joint model showed unusually large deformations in the cartilage adjacent to an empty defect [3]. Ex vivo studies demonstrated abnormally high strains that were mapped around empty focal chondral defects [4]. Computational modeling of focal defects located in regions of direct cartilage-cartilage contact also indicated supraphysiological strain levels [5]. The magnitude of these reported strains are associated with cell-mediated cartilage degradation, cell death, and even tissue failure [6]. Indeed, these regions of high contract stresses have been linked with symptomatic osteoarthritis in at-risk human patients [7]. These studies raise the question if the defect is filled with a mechanically supportive material, can degeneration be prevented within a loading environment?

The goals for this study were two-fold. First, we aimed to develop an ex vivo experimental model of a focal chondral defect that captures cartilage degeneration under physiological loading similar to that observed in an animal model, and enables the role of loading to be decoupled. To accomplish this goal, porcine focal chondral defects were created either in vivo in the knee of a pig or in explants of porcine osteochondral plugs. The latter was cultured under free swelling or subjected to compressive mechanical loading to emulate aspects of the physiological environment. We next evaluated the health of the adjacent cartilage when infilled with an in situ forming hydrogel and subjected to daily mechanical loads. Poly(ethylene glycol) (PEG) hydrogels were chosen for their tunability including achieving compressive moduli similar to that of articular cartilage [8-11]. Two hydrogel formulations were investigated that were softer or of similar stiffness to cartilage. In both studies, the cartilage adjacent to the defect was analyzed for loss of sulfated glycosaminoglycans (sGAGs). In the ex vivo studies, mechanical properties of the cartilage adjacent to the defect were analyzed by atomic force microscopy.

### 2. Materials and Methods

Detailed in Examples section.

### 3. Results

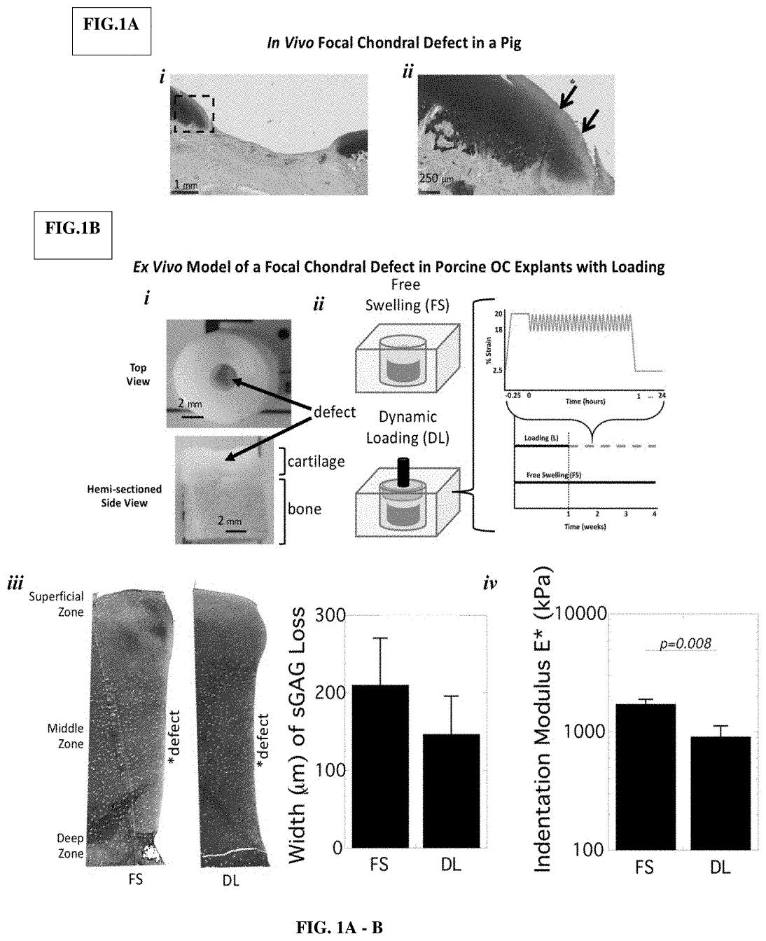

Tissue surrounding the empty focal chondral defect in vivo was evaluated four weeks post-injury (FIG. 1A). The defect remained largely empty with limited fibrous tissue formation adjacent to the calcified zone. The adjacent hyaline cartilage showed signs of fissures and an irregular border with the calcified zone. A region of reduced sGAGs that spanned a distance of approximately 200 um from the edge of the defect was evident. This finding indicates that within four weeks, substantial damage occurs to the tissue immediately surrounding the defect.

To create an ex vivo model, full depth focal chondral defects were created in explants of osteochondral plugs that accounted for ˜12% of the surface area (FIG. 18i). The explants were cultured for four weeks under free swelling or under dynamic compressive loading for one hour per day (FIG. 1Bii) Signs of degeneration in the cartilage adjacent to the defect were evident by reduced staining for sGAGs within the middle zone (FIG. 1Biii). The distance of degeneration from the defect's edge was 210 (60) μm under free swelling and, whose mean was lower, but not statistically significant at 150 (50) μm under loading. AFM assessment of cartilage modulus was performed in the middle zone of cartilage where sGAG degeneration was most pronounced. While histological assessment showed the greatest sGAG loss at the defect edge, the indented regions were placed centrally within each cartilage explant to avoid experimental variability associated with both edge effects and the steep sGAG gradient that was observed adjacent to the defect. This approach also gave an assessment of the quality of cartilage away from the defect that supports loads applied to the articular surface. The indentation modulus was 1700 (170) kPa under free swelling and decreased (p=0.008) by 47% under loading (FIG. 1Biv).

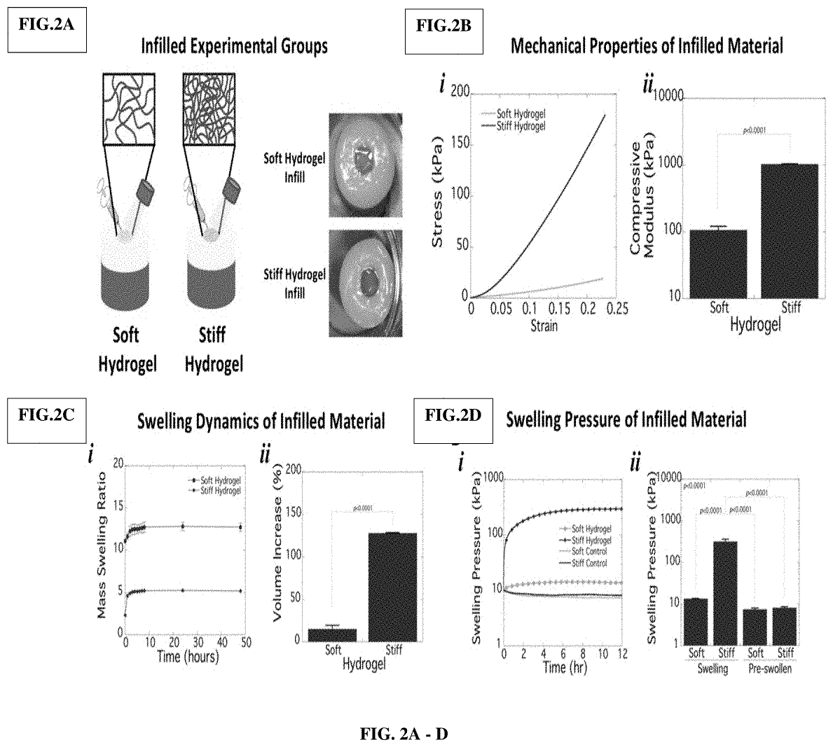

The defects were infilled with either a soft or stiff hydrogel. After infilling, the hydrogels adhered to the cartilage and remained in place after swelling (FIG. 2A). The hydrogels were first characterized on specimens alone (i.e., not within the defect). Representative stress-strain plots for soft and stiff hydrogels are shown along with the resulting compressive modulus of 100 kPa and 1 MPa for the soft and stiff hydrogels, respectively (FIG. 2B). Because the hydrogels are formed in situ in the defect, a swelling pressure is generated as the hydrogels reach equilibrium. The mass swelling ratio for the soft hydrogel increased from 11 to 13 over ˜eight hours and then did not significantly change thereafter, indicating that they had reached equilibrium (FIG. 2Ci). The mass swelling ratio for the stiff hydrogel increased from 2.3 to 5.2 over ˜eight hours and then remained constant (FIG. 2Ci). The volume increase from the polymerized state to equilibrium was 15% for the soft hydrogel and 130% for the stiff hydrogel (FIG. 2Cii). The swelling pressure increased over time corresponding with the temporal swelling response (FIG. 2Di). Contrarily, the pre-swollen hydrogels exhibited a small amount of stress relaxation. The maximum stresses were 13 kPa and 310 kPa for the soft and stiff hydrogels, respectively (FIG. 2Bii). Both were higher than the equilibrium stress of the pre-swollen hydrogels.

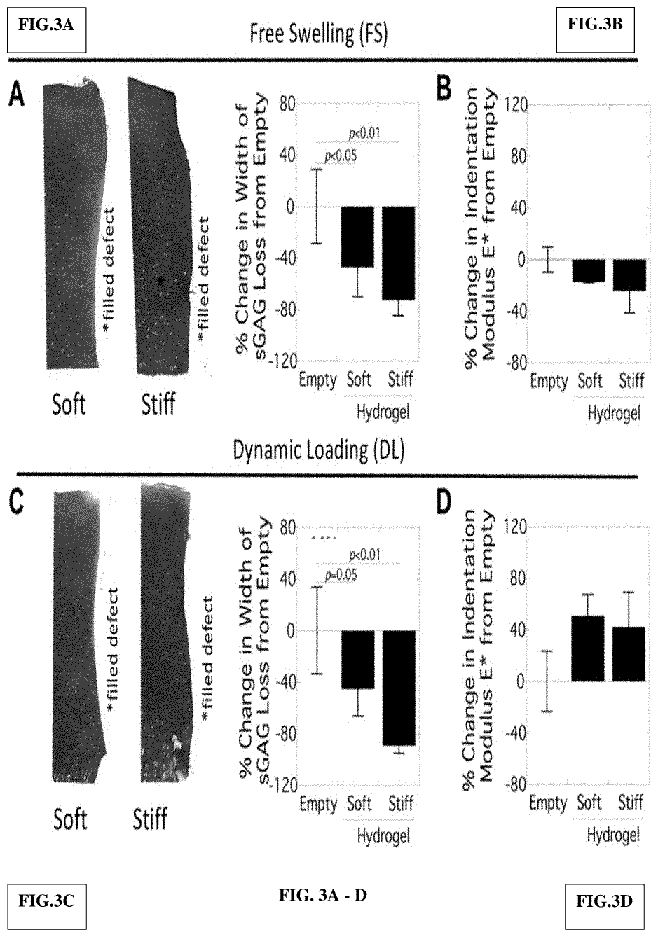

Under free swelling, the cartilage adjacent to the filled defects showed visibly less sGAG loss along the edge of the defect over the empty defect under free swelling (compare FIG. 3A to FIG. 1Biii). This distance was 110 (48) μm and 57 (25) μm for the soft and stiff infilled hydrogel, respectively. The width of sGAG loss decreased by 47% and 73% for the soft and stiff infilled hydrogel, respectively, from the empty defect (FIG. 3A). The indentation modulus was not affected by the infilled hydrogel. Under loading, there was also visibly less sGAG loss when compared to the empty defect (compare FIG. 3C to FIG. 1Biii). This distance was 80 (31) μm and 16 (9) μm for the soft and stiff infilled hydrogel, respectively. The width of sGAG loss decreased by 45% and by 89% for the soft and stiff infilled hydrogels, respectively, from the empty defect (FIG. 3C). A trend of increased indentation modulus was evident in the infilled hydrogels under loading, but this difference was not (p=0.068) statistically significant.

### 4. Discussion

This study demonstrates that cartilage adjacent to a chondral defect in vivo in porcine articular cartilage shows substantial signs of physical damage and tissue degeneration within four weeks post-injury. The ex vivo focal chondral defect also showed signs of cartilage degeneration adjacent to a defect. However, it did not capture the physical damage of the cartilage that was observed in vivo; a finding attributed to the absence of sliding ex vivo. The ex vivo model, however, enabled decoupling of the role of loading in cartilage degeneration and investigation into the prevention of degeneration by infilling the defect with a hydrogel. Our results show that degeneration was prevalent regardless of loading and that infilling the defect significantly reduced degeneration, with hydrogels of increased stiffness and swelling pressures having an even greater beneficial effect. Overall, this study provides evidence that degeneration occurs along the defect regardless of loading, but which can be prevented by infilling due to the effects of hydrogel swelling pressure and stiffness.

Our in vitro results indicate that degeneration, based on sGAG loss, is prevalent regardless of loading. When collagen fibers are damaged after an acute injury, the collagen network no longer resists the swelling pressures of proteoglycans, leading to cartilage swelling [17,18]. This effect was most pronounced ex vivo in the middle zone where sGAG concentration is highest [19]. This sGAG loss may be attributed to cell-mediated degradation where chondrocytes respond to increased cartilage swelling, and/or to physically-mediated degradation where swelling effectively increases porosity and diffusive transport. sGAGs are localized in large aggrecan aggregates that reach several microns [20]. It seems less likely that increased porosity could lead to loss of aggrecan aggregates alone without concurrent aggrecan degradation. This conjecture is supported by the observation that sGAG loss was not immediate, as infilling twenty-four hours post-injury reduced sGAG loss long-term. Moreover, studies have reported an increase in swelling in the middle zone without sGAG loss [17]. While mechanical injury can lead to physical loss within the first twenty-four hours, extended sGAG loss after injury occurs by enzyme-mediated degradation [21]. In vivo, sGAG loss was apparent throughout the cartilage thickness and was accompanied by evidence of mechanical damage. We surmise that ex vivo, sGAG loss observed along the middle zone is due to cell-mediated degradation resulting from localized tissue swelling that alters the chondrocyte environment. However, the in vivo environment is more complex with uniaxial loading combined with sliding and the presence of inflammatory mediators. The sGAG loss observed in vivo is likely due to both mechanical injury and cell-mediated degradation.

Simply infilling the defect with a hydrogel significantly reduced degeneration along the defect. This finding points to an intrinsic property of the hydrogel that is capable of partially protecting the tissue. As the hydrogels were formed directly in the defect, hydrogel expansion due to swelling is restricted and instead, a swelling pressure is generated at the adjacent tissue surfaces. Interestingly, both hydrogels reduced sGAG loss suggesting that swelling pressures of 13 kPa may be sufficient to minimize degeneration, but that higher swelling pressures (e.g., 310 kPa) may provide an even greater reduction in degeneration. This magnitude is similar to those reported during hydrogel degradation where swelling pressures increased from 50 to 800 kPa [22]. These swelling pressures may serve to physically prevent the adjacent tissue from swelling and thus minimize cartilage swelling-induced sGAG loss and/or provide mechanical signals to maintain homeostasis. For example, studies have reported positive effects on chondrocytes when cartilage explants are exposed to static hydrostatic pressures [23]. Our data suggest that swelling pressures generated by the hydrogel may provide a mechanical stimulus that protects cartilage.

The mechanical properties of the cartilage distant to the empty defect were adversely affected by loading. This finding suggests that damage to cartilage may be more pervasive than that observed by sGAG presence. Finite element modeling of a focal chondral defect in a whole joint under loading indicated that elevated principal stresses and strains can extend large distances from a defect [5]. Infilling the defect with the hydrogels did not significantly alter the mechanical properties when compared to their empty counterpart. The hydrogels used in this study exhibit largely elastic behavior with minimal stress relaxation behavior. While cartilage exhibits large stress relaxation under static loading, the time dependent properties become less pronounced under dynamic loading [24]. It remains to be determined if the infill material needs to recapitulate the time-dependent mechanical properties of cartilage to preserve the mechanical properties. It is also important to note that we did not control for the location from where the osteochondral explants were taken in the joint, which may introduce variability across specimen.

In summary, the ex vivo model captures sGAG degeneration along the edge of a defect, similar to that which was observed in vivo. This model indicates that the sGAG loss appears to be largely independent of loading, but can be prevented by infilling with a swelling hydrogel. While the ex vivo model does not capture the full complexity of the joint environment (e.g., shear loading), it allows for the investigation of the defect in a more controlled environment and the ability to decouple the effects due to an applied uniaxial compressive load. Our ex vivo dynamic mechanical loading paradigm may then serve as a first step towards identifying infilled materials that can minimize tissue degeneration and maintain the mechanical properties in the cartilage surrounding the defect. Pro-inflammatory cytokines could also be introduced into the ex vivo model to simulate an inflammatory joint. Testing in vivo within the joint environment is ultimately critical to establish whether any infill material can also protect against the shear forces that result in mechanical damage.

### EXAMPLES

The following examples are provided in order to demonstrate and further illustrate certain preferred embodiments and aspects of the present invention and are not to be construed as limiting the scope thereof.

### Example 1

### In Vivo Porcine Studies of Focal Chondral Defect.

All procedures were approved by the Institutional Animal Care and Use Committee (IACUC) of the Massachusetts General Hospital. All anesthetics, antibiotics, and analgesics were obtained from Patterson Veterinary Supply, Fort Devens, Mass. Female Yorkshire swine were sedated with an intramuscular injection Telazol (4.4 mg/kg), Xylazine (2.2 mg/kg) and atropine (0.04 mg/kg), intubated, and anesthesia was maintained with inhaled Isofluorane at 1-5% titration. A single dose of Cefazolin 40 mg/kg IV was administered prior to incision. Buprenorphine (0.01-0.05 mg/kg IM) was given 30 minutes prior to surgery. The area around the stifle joint was washed with surgical scrub followed with betadine and sterile draped. A paramedian incision was made and the patella displaced laterally to allow for visualization of the trochlear groove. Four, 6 mm defects were made in the cartilage down to the underlying subchondral bone and hemostasis was achieved. The patella repositioned and stabilized in place using two, number 2 nylon sutures. The capsule, muscle and skin were closed. Fentanyl patch (1-4 μg/kg/hr) was applied and kept in place for 72 hours for analgesia. Animals were euthanized at four weeks and defects processed by histology.

### Example 2

Culture of Osteochondral Plugs with Chondral Focal Defects.

Osteochondral explants were obtained from the trochlear groove and femoral condyle of a 3-month-old female Yorkshire swine using a biopsy punch (8.5 mm diameter×10 mm height). Full thickness focal chondral defects (3 mm diameter) were created centrally without disrupting the underlying bone (total height of ˜2-3 mm). Explants were cultured individually in 24-well plates for four weeks under free swelling or under free swelling for one week followed by three weeks of unconfined dynamic compression in custom bioreactors (n=4) [8,12]. Explants were compressed (0.1 mm min⁻¹) to 20% strain and held for 15 minutes, then subjected to sinusoidal dynamic compression at 2% peak-to-peak strain at 1 Hz for one hour [13], and finally cultured under a constant 2.5% strain for 23 hours. This protocol was repeated five days each week. Explants were cultured in Dulbecco's Modified Eagle Medium (DMEM) with 10% fetal bovine serum, 0.05 mg ml⁻¹ 1-ascorbic acid, 0.4 mM L-proline, 0.1nM non-essential amino acids, 0.01 M HEPES, 0.02 mg ml⁻¹ gentamicin, 4 mM glutaGRO, 100 U ml⁻¹ penicillin, and 50 μg ml⁻¹ streptomycin. Medium was changed every 2-3 days.

### Example 3

### Hydrogel Formation and In Situ Filling of Focal Defects.

Poly(ethylene glycol) dimethacrylate (PEGDM) was synthesized from PEG (4600 g mol⁻¹) and methacrylic anhydride via microwave methacrylation [14]. Hydrogel precursors consisted of 10 (g/g) % (soft) or 40 (g/g) % (stiff) PEGDM in phosphate buffered saline (PBS, pH 7.4) with 0.05% (g/g) photoinitiator Irgacure 2959 (12959) and were polymerized under 352nm light at 5 mW cm⁻² for 10 minutes. For infilling, the defect was dried with 0.2 μm filtered CO₂ for 1 minute. Approximately 50 μl of filter-sterilized precursor solution was injected into each defect and photopolymerized. Filled explants were cultured (n=4 per group) as described previously.

### Example 4

### Mechanical Characterization of Hydrogel.

Compressive modulus testing: equilibrium swollen hydrogels (n=3-4) of 3 mm diameter, 3 mm height in PBS were strained (0.5 mm min⁻¹) in a mechanical tester (MTS Insight II) and compressive modulus determined from the linear region of the stress strain curve between 15 and 22%. Equilibrium mass swelling testing: hydrogels (n=3) of 5 mm diameter, 2.5 mm height were immediately weighed, placed in de-ionized water and mass measured every hour, for eight hours, and at 24 and 48-hours. Hydrogels were lyophilized and their dry polymer mass measured. Equilibrium mass swelling ratio (swollen/dry polymer masses) was determined. Swelling pressure experiments: soft and stiff hydrogels (n=3 per group) were first swollen in de-ionized water for 24 hours (i.e., pre-swollen control) and then placed or immediately placed into a sample cup with de-ionized water in a mechanical tester (BOSE Electroforce TestBench, Model 66-601B). A pre-load of 0.2 N was applied and load required to maintain the platen's initial position was recorded every two seconds for 24-hours. Load was normalized to the initial area of the hydrogel.

### Example 5

Histology

After 4 weeks, the cartilage was cut in half vertically. One-half was prepared for histology by removing the underlying bone. The sample was fixed in 10% formalin for two days at room temperature, embedded in paraffin and processed following standard protocols. Sections (20 μm) were stained for sulfated glycosaminoglycans (sGAGs) by Safranin O/Fast green and imaged at 100× by light microscopy (Zeiss Pascal, Olympus DP70). Quantitative analysis of sGAG loss was performed (NIH Image J) by measuring the width of degenerated tissue, defined by an absence of red stain. A total of n=10 line measurements were made perpendicular to the defect's edge per image for four images per side and two sides per specimen. All measurements for each specimen were averaged (n=4 per group).

### Example 6

Nanomechanical Analysis.

The other half of osteochondral explants (n=3 per group), designated for mechanical property assessment, was stored at −80° C. Each specimen was thawed immediately prior to testing. Slices (200 μm) spanning the entire cartilage thickness were obtained using a vibratome, fixed to a glass slide using cyanoacrylate, submerged in PBS, and mounted on the stage of an atomic force microscope (AFM) (Keysight 5500 AFM). One sample (free swelling, soft infill group) was contaminated with cyanoacrylate and removed from analysis. Cantilevers with a nominal stiffness of 16 N/m determined using Sader's method [15] were affixed with a colloidal probe with a nominal diameter of 25 μm determined by optical microscopy. Cantilever deflection sensitivity was re-calibrated immediately prior to testing. A minimum of 80 indents were performed in the middle zone of each slice and centrally placed at half width using a peak voltage corresponding to an approximate peak force of 500 nN and a piezo displacement rate of 50 μm/s, which was chosen to avoid poroelastic relaxation during loading. Custom MATLAB script was used to convert voltage vs. piezo displacement data into load (μN) vs. indentation depth (nm). Data were corrected for the point of contact [16] and a simple Hertzian analysis was used to determine indentation modulus, E*. The indentation modulus for all indents measured in a given sample were averaged to determine an overall E*.

### Example 7

Statistical Analysis.

Data are reported as mean with standard deviation as error bars or parenthetically in the text. Statistical analysis was performed using Real Statistics add-on for Excel. Statistical analyses included unpaired two-sample t-test assuming equal variance, an one-way ANalysis Of VAriance (α=0.05), or two-way ANOVA (α=0.05). Data were confirmed to be normally distributed and exhibit a homogeneous variance. Follow-up analyses were performed using a Tukey's post-hoc. P≤0.05 was considered statistically significant.

### Example 8

Supporting Data: Tunability of Swelling.

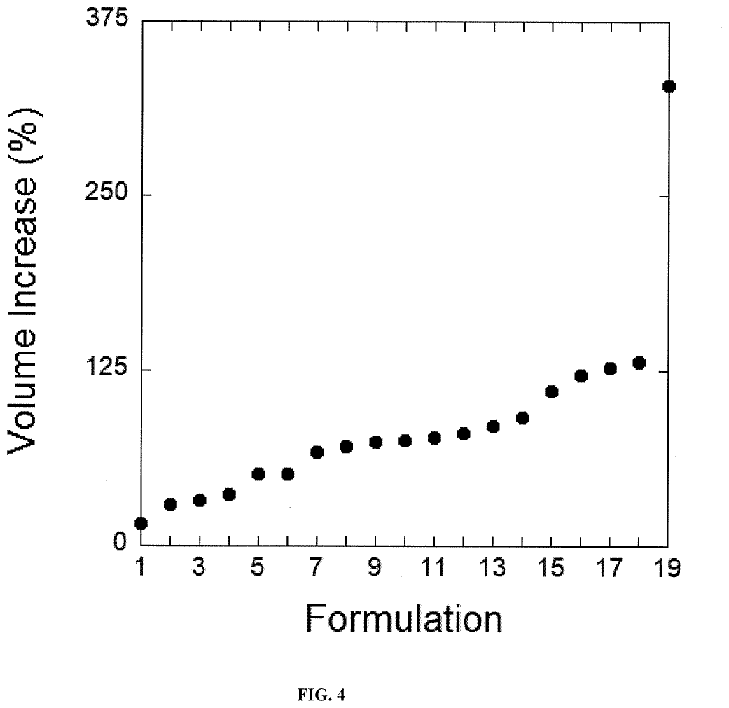

The present invention system demonstrates a range of tunable synthetic hydrogels. Modification of the hydrogel provides a control over the % volume increase of the hydrogel which, in turn, influences the swelling pressure. The various formulations 1-19 are described in Table 1 below. The performance of the formulations 1-19 for swelling (as measured in volume increase %) are shown in FIG. 4.

TABLE 1

| | | |
|---|---|---|
| Formulation | Polymer wt % (MW PEG) | Thiol:Ene |

| | | | |
|---|---|---|---|
| 1 | 5 | (20 kDa) | 0.95 |
| 2 | 10 | (10 kDa) | 1.00 |
| 3 | 10 | (10 kDa) | 0.90 |
| 4 | 10 | (10 kDa) | 0.65 |
| 5 | 8 | (20 kDa) | 0.95 |
| 6 | 7 | (10 kDa) | 0.90 |
| 7 | 15 | (10 kDa) | 0.75 |
| 8 | 9 | (20 kDa) | 0.90 |
| 9 | 9 | (20 kDa) | 1.00 |
| 10 | 10 | (10 kDa) | 0.50 |
| 11 | 10 | (20 kDa) | 0.95 |
| 12 | 15 | (10 kDa) | 0.90 |
| 13 | 20 | (10 kDa) | 0.90 |
| 14 | 9 | (20 kDa) | 0.70 |
| 15 | 12 | (20 kDa) | 0.95 |
| 16 | 25 | (10 kDa) | 1.00 |
| 17 | 9 | (20 kDa) | 0.50 |
| 18 | 13 | (20 kDa) | 0.95 |
| 19 | 9 | (20 kDa) | 0.30 |

### Example 9

Composite Structure.

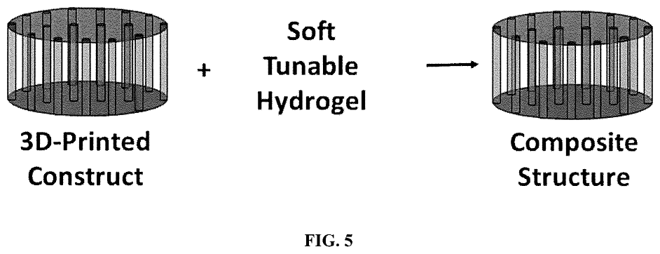

The present invention also contemplates the integration of 3D-printed construct to provide support for physiological loads at injury site in combination with the tunable synthetic hydrogel. The present invention soft hydrogel can be tailored to provide the necessary swelling pressure to support the tissue surrounding the focal defect. The hydrogel formulation can be adjusted to control the degree of swelling and therefore the swelling force exerted on the surrounding tissue. In addition, the precursor solution can be tailored to maximize efficacy for a given injury site. For example, cartilage biomimetic cues can be added to enhance swelling and compatibility within the defect zone. An example of the integration of 3D-printed construct to provide support for physiological loads at injury site in combination with the tunable synthetic hydrogel is shown in FIG. 5.

### Example 10

Composite Structure for Protecting Surrounding Tissue.

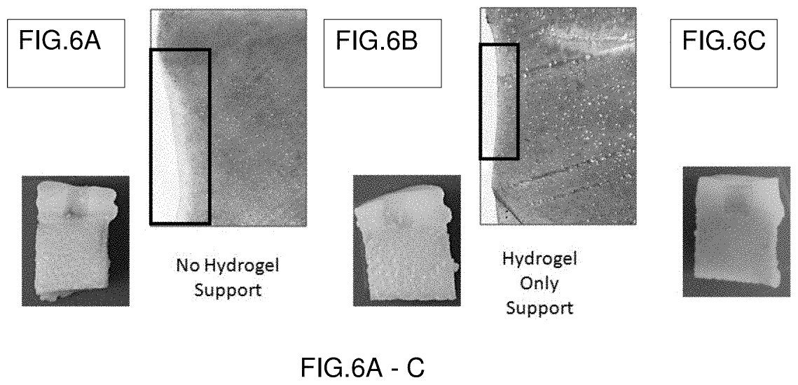

The present invention also contemplates the use of an integrated 3D-printed construct to provide support for physiological loads at injury site in combination with the tunable synthetic hydrogel. To examine the various compositions, porcine plugs with focal defect were used to examine the effects of an alternate hydrogel formulation and 3D-printed scaffold on the area surrounding the focal defect. FIG. 6A shows a porcine plugs with focal defect which was not treated with a hydrogel support. FIG. 6B shows a porcine plugs with focal defect which was treated with a hydrogel only support. FIG. 6C shows a porcine plugs with focal defect which was treated with a 3D-scaffold and hydrogel support. Preliminary results indicate that the composite structure resulted in the most effective protection when samples were exposed to a regular compressive force.

Thus, specific compositions and methods of prevention of cartilage degeneration surrounding focal chondral defects have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

### REFERENCES

[1] W. Widuchowski, J. Widuchowski, T. Trzaska, Articular cartilage defects: Study of 25,124 knee arthroscopies, Knee. 14 (2007) 177-182.
[2] J. H. Guettler, C. K. Demetropoulos, K. H. Yang, K. A. Jurist, Osteochondral deflects in the human knee—Influence of defect size on cartilage rim stress and load redistribution to surrounding cartilage, Am. J. Sports Med. 32 (2004) 1451-1458.
[3] J. P. Braman, J. D. Bruckner, J. M. Clark, A. G. Norman, H. A. Chansky, Articular cartilage adjacent to experimental defects is subject to atypical strains., Clin. Orthop. (2005) 202-207.
[4] K. R. Gratz, B. L. Wong, W. C. Bae, R. L. Sah, The Effects of Focal Articular Defects on Cartilage Contact Mechanics, J. Orthop. Res. 27 (2009) 584-592.
[5] M. S. Venalainen, M. E. Mononen, J. Salo, L. P. Rasanen, J. S. Jurvelin, J. Toyras, T. Viren, R. K. Korhonen, Quantitative Evaluation of the Mechanical Risks Caused by Focal Cartilage Defects in the Knee., Sci. Rep. 6 (2016) 37538.
[6] T. M. Quinn, R. G. Allen, B. J. Schalet, P. Perumbuli, E. B. Hunziker, Matrix and cell injury due to sub-impact loading of adult bovine articular cartilage explants: effects of strain rate and peak stress., J. Orthop. Res. Off. Publ. Orthop. Res. Soc. 19 (2001) 242-249.
[7] N.A. Segal, D. D. Anderson, K. S. Iyer, J. Baker, J. C. Tomer, J. A. Lynch, D. T. Felson, C. E. Lewis, T. D. Brown, Baseline articular contact stress levels predict incident symptomatic knee osteoarthritis development in the MOST cohort., J. Orthop. Res. Off. Publ. Orthop. Res. Soc. 27 (2009) 1562-1568.
[8] G. D. Nicodemus, S. J. Bryant, The role of hydrogel structure and dynamic loading on chondrocyte gene expression and matrix formation, J. Biomech. 41 (2008) 1528-1536.
[9] B. V. Slaughter, S. S. Khurshid, O. Z. Fisher, A. Khademhosseini, N. A. Peppas, Hydrogels in Regenerative Medicine, Adv. Mater. Deerfield Beach Fla. 21 (2009) 3307-3329.
[10] S. Bryant, K. Anseth, Photopolymerization of Hydrogel Scaffolds, in: Scaffolding Tissue Eng., CRC Press, 2005: pp. 71-90.
[11] J. L. Ifkovits, J. A. Burdick, Review: photopolymerizable and degradable biomaterials for tissue engineering applications, Tissue Eng. 13 (2007) 2369-2385. doi:10.1089/ten.2007.0093.
[12] I. Villanueva, S. K. Gladem, J. Kessler, S. J. Bryant, Dynamic loading stimulates chondrocyte biosynthesis when encapsulated in charged hydrogels prepared from poly(ethylene glycol) and chondroitin sulfate, Matrix Biol. J. Int. Soc. Matrix Biol. 29 (2010) 51-62.
[13] R. L. Y. Sah, Y. J. Kim, J. Y. H. Doong, A. J. Grodzinsky, A. H. K. Plaas, J. D. Sandy, Biosynthetic response of cartilage explants to dynamic compression, J. Orthop. Res. 7 (1989) 619-636.
[14] S. Lin-Gibson, S. Bencherif, J. A. Cooper, S. J. Wetzel, J. M. Antonucci, B. M. Vogel, F. Horkay, N. R. Washburn, Synthesis and Characterization of PEG Dimethacrylates and Their Hydrogels, Biomacromolecules. 5 (2004) 1280-1287.
[15] J. E. Sader, J. W. M. Chon, P. Mulvaney, Calibration of rectangular atomic force microscope cantilevers, Rev. Sci. Instrum. 70 (1999) 3967-3969. doi:10.1063/1.1150021.
[16] S.L. Guo, B. B. Akhremitchev, Packing density and structural heterogeneity of insulin amyloid fibrils measured by AFM nanoindentation, Biomacromolecules. 7 (2006) 1630-1636.
[17] A. Maroudas, M. Venn, Chemical composition and swelling of normal and osteoarthrotic femoral head cartilage. II. Swelling., Ann. Rheum. Dis. 36 (1977) 399-406.
[18] M. Nickien, A. Thambyah, N. D. Broom, How a decreased fibrillar interconnectivity influences stiffness and swelling properties during early cartilage degeneration, J. Mech. Behay. Biomed. Mater. 75 (2017) 390-398.
[19] M. Venn, A. Maroudas, Chemical composition and swelling of normal and osteoarthrotic femoral head cartilage. I. Chemical composition., Ann. Rheum. Dis. 36 (1977) 121-129.
[20] M. Cornelissen, M. Dewulf, G. Verbruggen, P. Hellebuyck, A. M. Malfait, L. De Ridder, E. M. Veys, Size distribution of native aggrecan aggregates of human articular chondrocytes in agarose., In Vitro Cell. Dev. Biol. Anim. 29A (1993) 356-358.
[21] M. A. DiMicco, P. Patwari, P. N. Siparsky, S. Kumar, M. A. Pratta, M. W. Lark, Y. J. Kim, A. J. Grodzinsky, Mechanisms and kinetics of glycosaminoglycan release following in vitro cartilage injury, Arthritis Rheum. 50 (2004) 840-848.
[22] J. Hrib, J. Sirc, P. Lesny, R. Hobzova, M. Duskova-Smrckova, J. Michalek, R. Smucler, Hydrogel tissue expanders for stomatology. Part I. Methacrylate-based polymers, J. Mater. Sci.-Mater. Med. 28 (2017) 12.
[23] B. D. Elder, K. A. Athanasiou, Hydrostatic pressure in articular cartilage tissue engineering: from chondrocytes to tissue regeneration., Tissue Eng. Part B Rev. 15 (2009) 43-53.
[24] Z. Trad, A. Barkaoui, M. Chafra, J. M. R. S. Tavares, FEM Analysis of the Human Knee Joint: A Review, in: Fem Anal. Hum. Knee Jt. Rev., 2018: pp. 1-79.
[25] Hoyle, C. E. et al. (2004) “Thiol-Enes: Chemistry of the Past with Promise for the Future,” J. Polym. Sci. A Polym. Chem. 42(21), 5301-5338.
[26] Lin, C.-C. et al. (2011) “PEG Hydrogels Formed by Thiol-Ene Photo-Click Chemistry and Their Effect on the Formation and Recovery of Insulin-Secreting Cell Spheroids,” Biomaterials 32(36), 9685-9695.

We claim:

1. A method for treating a chondral defect in a patient in need thereof comprising administering to the patient a therapeutically effective amount of a hydrogel composition within said chondral defect.

2. The method of claim 1, wherein said treating prevents cartilage diseases.

3. The method of claim 2, wherein said cartilage disease comprises joint degeneration.

4. The method of claim 2, wherein said cartilage disease comprises osteoarthritis.

5. The method of claim 1, wherein said chondral defect is due to an injury.

6. The method of claim 1, wherein said treating reduces the effects of cartilage degneration.

7. The method of claim 1, wherein said treating comprises infilling said chondral defect with said hydrogel composition.

8. The method of claim 1, wherein said hydrogel composition produce swelling pressures of 13 and 310 kPa.

9. The method of claim 7, wherein said hydrogel composition produce swelling pressures of 13 and 310 kPa.

10. The method of claim 1, wherein said treating reduces the loss of sulfated glycosaminoglycans surrounding said chondral defect.

11. The method of claim 1. wherein said hydrogel is polymerized in silo.

12. The method of claim 1. wherein said hydrogel comprises Poly(ethylene glycol) (PEG).

13. The method of claim 12. wherein said hydrogel comprises Poly(ethylene glycol) dimethacrylate.

10. The method of claim 1, wherein said treating comprises integration of a 3D-scaffold into said chondral defect in addition to said hydrogel composition.

15. The method of claim 1, wherein said hydrogel is created by photopolymerization of mixtures of multifunctional thiols and enes.

16. The method of claim 15, wherein said hydrogel comprises a polymer weight percentage 13% (20 kDa) (MW PEG) with a ratio of thiol:ene of 0.95.

17. The method of claim 15, wherein said hydrogel comprises a polymer weight percentage between 5-10% 10-20 kDa MW PEG with a ratio of thiol:ene between 0.30-0.95.

18. The method of claim 15, wherein said hydrogel comprises a polymer weight percentage between 10-25% 10-20 kDa MW PEG with a ratio of thiol:ene between 0.50-1.00.
